(19) United States
(12) Patent Application Publication
(10) Pub. No.: US 2016/0287726 A1
(43) Pub. Date: Oct. 6, 2016

(54) CATIONIC CONTRAST AGENTS AND METHODS OF USING THE SAME

(71) Applicant: The Trustees of the University of Pennsylvania, Philadelphia, PA (US)

(72) Inventors: Andrew Tsourkas, Bryn Mawr, PA (US); Kido Nwe, Philadelphia, PA (US)

(21) Appl. No.: 15/076,041

(22) Filed: Mar. 21, 2016

Related U.S. Application Data
(60) Provisional application No. 62/143,401, filed on Apr. 6, 2015.

(51) Int. Cl. A61K 49/10

(52) U.S. Cl. CPC A61K 49/108; A61K 49/106

(57) ABSTRACT

Gadolinium complexes for use as contrast agents, and methods for making and using the gadolinium complexes, are described. The contrast agent complexes preferably have a net positive charge, and can electrostatically interact with glycosaminoglycans to improve the delineation of fine tears within cartilage, detection of cartilage degeneration, or assessment of cartilage thickness, morphology, or glycosaminoglycan content via magnetic resonance imaging.

### CROSS REFERENCE TO RELATED APPLICATIONS

This application claims priority to U.S. Provisional Application No. 62/143401, entitled CATIONIC CONTRAST AGENTS AND METHODS OF USING THE SAME, filed Apr. 6, 2015, the contents of which are incorporated by reference herein in their entirety.

### STATEMENT REGARDING FEDERALLY SPONSORED RESEARCH

This invention was made with government support by the National Institutes of Health NIBIB R01EB012065 (AT) and NCI R01CA157766 (AT). The government has certain rights in the invention.

### FIELD OF THE INVENTION

Embodiments of the present invention relate to contrast agents and methods of using the contrast agents.

### BACKGROUND OF THE INVENTION

Diagnostic imaging of joints usually begins with a radiographic evaluation, which can help detect obvious sources of disease such as advanced arthritis, tumor, dislocation, or impingement. However, X-ray and computed tomography (CT) scans cannot detect tears in cartilage and other soft tissues. The early diagnosis of a tear in the fibrocartilaginous knee meniscus is considered important in order to minimize the potential for the development of significant erosion of the articulating surfaces, with cartilage defects and meniscus tears potentially preceding and possibly leading to additional chondral damage.

Conventional magnetic resonance (MR) imaging does demonstrate some intra-articular defects such as large deep cartilage deficiencies or avascular necrosis and better soft tissue depiction. However, despite generally being favored over CT, MR still has a lower than desirable sensitivity at demonstrating tears in fibrocartilaginous tissues in the joint (such as the knee meniscus) and in identifying partial thickness cartilage defects in the articulating surfaces. The diagnostic accuracy of MR imaging is improved by the intra-articular injection of gadolinium (Gd)-based contrast agents, i.e., magnetic resonance arthrography (MRA). For example, a meta-analysis covering 881 hips across nineteen studies revealed that MRA led to a statistically significant improvement in the sensitivity of detecting acetabular labral tears compared with conventional MR, 87% vs. 66%. However, there is fairly broad variability between these studies, with sensitivity ranging from 60% to 100% and specificity ranging from 44% to 100%. In a separate study that examined various joint pathologies, the accuracy of MRA for labral, acetabular chondrosis, and femoral chondrosis and impingement lesions were only 85, 79, 59, and 82%, respectively, when images were read by musculoskeletal radiologists. The accuracy rates for general radiologists were significantly lower, 7, 28, 52, and 59%, respectively, further supporting the difficulty in making a correct diagnosis. Detecting joint pathologies becomes even more challenging following arthroscopic repair. It is important to note that poor/variable sensitivity and specificity is not limited to the hip, but is seen with the various major joints including the shoulder, elbow, knee and wrist.

This variability of MRA-based diagnoses of cartilage tears may stem from the use of anionic contrast agents, such as Magnevist®, also known as Gd-DTPA (Gd-diethylenetriaminepentacetate), shown below:

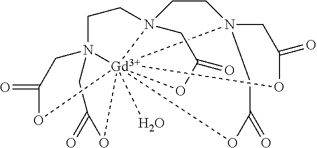

Magnevist® has a net charge of −2 (FIG. 1A). Given their anionic nature, it is believed that these agents experience electrostatic repulsion with major constituents of articular cartilage and other fibrocartilages in the joint that have a high concentration of negatively charged glycosaminoglycans (GAGs). Indeed, this interaction at the tissue-fluid interface may predispose the agents to poor penetration into narrow crevices, making it difficult to identify fine features, tears, and thinning cartilage (FIG. 1C). Thus, there remains a need for contrast agents that have increased electrostatic interactions with GAGs, and thereby have an improved ability to distinguish cartilage from surrounding tissue via magnetic resonance imaging, particularly via magnetic resonance arthrography.

### SUMMARY OF THE INVENTION

An embodiment of the present invention relates to a contrast agent complex (preferably a gadolinium complex) having a net positive charge, whereby the number of protons exceeds the number of electrons. For example, gadolinium ion (Gd³⁺) possesses a positive charge of three; to create a gadolinium complex with a net positive charge, the chelating agent will preferably have a net negative charge of −2 or less.

Another embodiment of the present invention relates to a polyazamacrocyclic compound having structure (I):

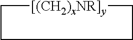

where each x is 2 or 3, y is 3 or 4, R is —CH(CO₂X)R′, X is H or an alkali metal, and R′ is a primary amine-functionalized substituent. A preferred embodiment of this compound is:

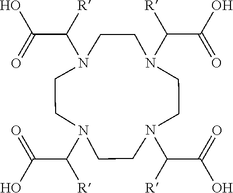

where R′ is a primary amine-functionalized substituent.

Another embodiment of the present invention relates to a compound having structure (I) complexed with a metal (preferably gadolinium). Such a complex is referred to herein as a contrast agent complex. A preferred embodiment of the contrast agent complex is:

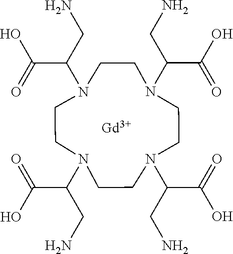

Another embodiment of the present invention relates to contrast agent composition comprising a contrast agent complex, a carrier (preferably a liquid carrier), and one or more optional additives.

Another embodiment of the present invention relates to a method of using a contrast agent complex comprising administering the contrast agent complex to a subject (e.g., by injection into a subject's joint). Preferably, the method further comprises imaging the subject's joint. According to an exemplary embodiment, magnetic resonance arthrography (MRA) is utilized to image the subject's joint.

### BRIEF DESCRIPTION OF THE DRAWINGS

FIG. 1 provides (A) a depiction of Magnevist (Gd-DTPA)²⁻, an anionic contrast agent, (B) a depiction of (Gd-DOTA-Am4)²⁺, a cationic contrast agent complex, (C) a theoretical depiction of electrostatic repulsion between an anionic contrast agent and negatively charged constituents of fibrocartilaginous tissue, demonstrating the poor penetration into narrow crevices, and (D) a theoretical depiction of electrostatic interaction between a cationic contrast agent and negatively charged constituents of fibrocartilaginous tissue, demonstrating efficient penetration into narrow crevices.

FIG. 2 provides longitudinal relaxivity (r₁) measurements of (A) Complex A (an embodiment of the present invention, also referred to as “Gd-DOTA-Am4”) and (B) Magnevist® (Gd-DTPA).

FIG. 3 provides an assessment of Gd-ligand stability: (A) transmetallation of 2.5 mM Complex A (an embodiment of the present invention) (), Gd-DTPA (▴), and Gd-DOTA (▪) in the presence of 2.5 mM ZnCl₂ was monitored by calculating the relaxation ratio R1(t)/R1(0) as a function of time (all transmetallation assays were performed in fetal bovine serum at pH 7.4 and 37° C.); (B) demetallation of 2.5 mM Complex A (), Gd-DTPA (▴), and Gd-DOTA (▪) in 1M HCl was monitored by measuring the T1 relaxation time as a function of exposure time; the T1 relaxation time of Gd(NO₃)₃ (♦) was also measured.

FIG. 4 provides (A) titration curve for Gd-DOTA-Am4, (B) the zeta potential of various compounds with known charges at pH 7.4, compared with Gd-DOTA-Am4.

FIG. 5 provides an assessment of the electrostatic interactions of Gd-DOTA-Am4, Gd-DOTA, and Gd-DTPA with GAG-rich cartilage based on T1 relaxation time.

FIG. 6 provides (A) T1-weighted MR images of bovine meniscus explants with “injury,” following incubation with Complex A (an embodiment of the present invention, also referred to as “Gd-DOTA-Am4”), Gd-DTPA, or Saline. Yellow boxes indicate ROIs. One MR imaging plane from each analyzed sample is shown. (B-D) Waterfall plots of signal intensity, following background subtraction, for the ROIs shown in subfigure A, Set 1. (E) The average signal intensity, normalized by the length of the defect, was calculated for all three sets of explants. Error bars indicate standard deviations. **p<0.001, ***p<0.0001.

FIG. 7 provides another set of the T1-weighted MR images depicting bovine meniscus explants with “injury” following incubation Complex A (an embodiment of the present invention, also referred to as “Gd-DOTA-Am4”), Gd-DTPA, or saline.

FIG. 8 provides images of monolayers of isolated bovine meniscal fibrochondrocytes (MFCs) exposed to basal media containing saline, Gd-DTPA, or Gd-DOTA-Am4 to depict cell morphology, viability, and proliferation over a 24-hour period.

FIG. 9 provides (A) images of cartilage and meniscus of an intact bovine femorotibial explant injected intra-articularly with saline, Gd-DTPA, or Gd-DOTA-Am4, and (B) a graphical depiction of the area fractions of live and dead cells in the cartilage and meniscus after 3 hours.

### DETAILED DESCRIPTION OF THE INVENTION

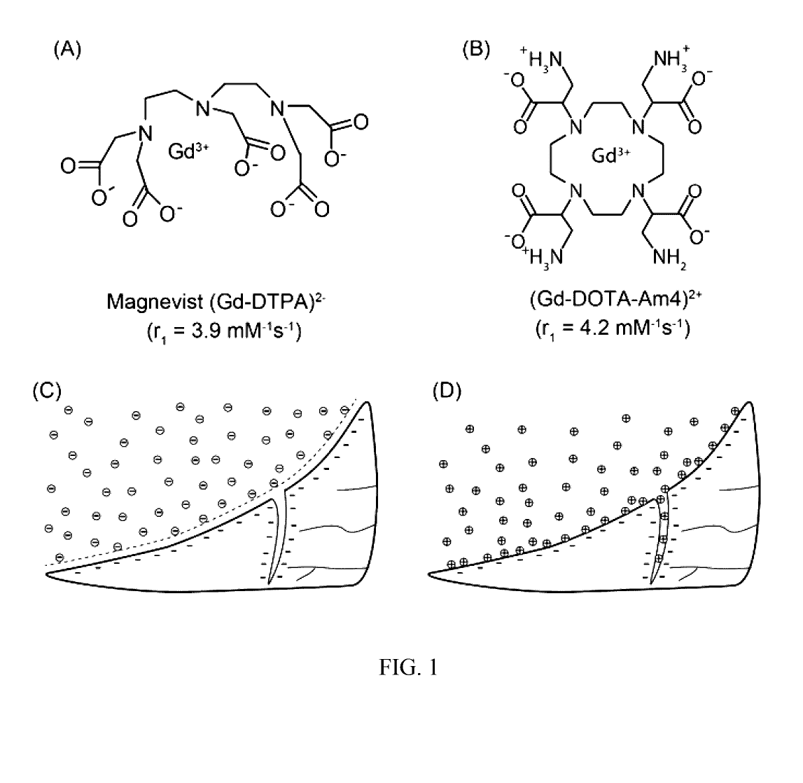

Recent studies have shown that the introduction of cationic charges onto computed tomography (CT) contrast agents may improve the ability to distinguish cartilage from surrounding tissue via CT, due to the increase in electrostatic interactions with glycosaminoglycans (GAGs). However, no cationic magnetic resonance (MR) contrast agents have previously been developed or evaluated (e.g., for magnetic resonance arthrography or MRA). It has been found that a cationic contrast agent complex, for example Gd-DOTA-Am4 (FIG. 1B), may interact with GAGs electrostatically to penetrate more efficiently into fine tears (FIG. 1D), allowing the tissue-fluid interface and cartilaginous tissue defects to be more clearly visualized.

Embodiments of the present invention relate to magnetic resonance contrast agents with a net positive charge, which have the ability to improve the delineation of fine tears in soft tissue (e.g., cartilage tissue). It is believed that the contrast agent complexes of the present invention interact electrostatically with charged glycosaminoglycans (GAGs) to improve the diagnostic accuracy of magnetic resonance imaging, particularly MRA.

Embodiments of the present invention relate to a polyazamacrocyclic compound having structure (I) (referred to herein as “Compound I”):

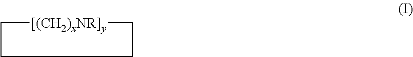

where x is 2 or 3, y is 3 or 4, R is —CH(CO₂X)R′, X is H or an alkali metal, and R′ is a primary amine-functionalized substituent.

According to particular embodiments, the primary amine-functionalized substituent contains two or more primary amine groups. According to additional embodiments, the primary amine-functionalized substituent contains a primary amine group at the terminus of an organic moiety. The organic moiety may contain, for example, a backbone chain of from 1 to 10 atoms. According to particular embodiments, the backbone chain contains at least one carbon atom and, optionally, at least one heteroatom selected from S, O or N.

According to particular embodiments, the primary amine-functionalized substituent R′ corresponds to structure (II):

—CH₂(R″)NH₂ (II)

wherein R″ is a covalent bond or a divalent organic moiety. The divalent organic moiety may contain, for example, a backbone chain of from 1 to 9 atoms. According to particular embodiments, the backbone chain consists of atoms selected from the group consisting of C, S, O and N. According to particular embodiments, R″ is a covalent bond. According to alternative embodiments, R″ is —NHSO₂—, —OC(═O)CH₂CH₂—, or —NHC(═O)CH(NH₂)—(CH₂)₄—.

According to particular embodiments, x is 2 and y is 4. For example, the compound having structure (I) may have the following structure:

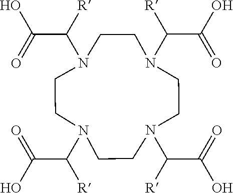

where R′ is a primary amine-functionalized substituent as defined above, in accordance with all of the embodiments described above.

As described above in accordance with particular embodiments, the compound having structure (I) has the following structure (Ia) (referred to herein as “Compound Ia”):

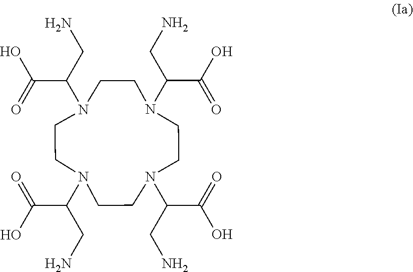 [SMILES: NCC(C(=O)O)N1CCN(C(CN)C(=O)O)CCN(C(CN)C(=O)O)CCN(C(CN)C(=O)O)CC1]

As described above in accordance with particular embodiments, the compound having structure (I) has the following structure (Ib) (referred to herein as “Compound Ib”):

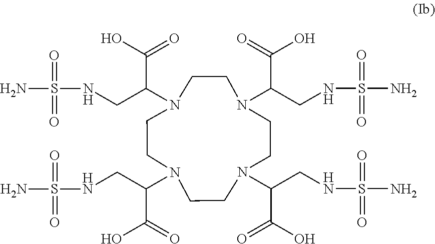 [SMILES: NS(=O)(=O)NCC(C(=O)O)N1CCN(C(CNS(N)(=O)=O)C(=O)O)CCN(C(CNS(N)(=O)=O)C(=O)O)CCN(C(CNS(N)(=O)=O)C(=O)O)CC1]

As described above in accordance with particular embodiments, the compound having structure (I) has the following structure (Ic) (referred to herein as “Compound Ic”):

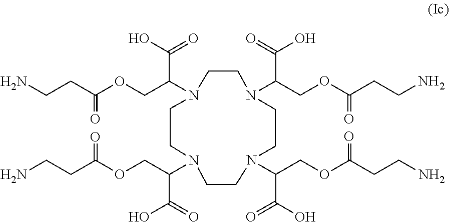 [SMILES: NCCC(=O)OCC(C(=O)O)N1CCN(C(COC(=O)CCN)C(=O)O)CCN(C(COC(=O)CCN)C(=O)O)CCN(C(COC(=O)CCN)C(=O)O)CC1]

As described above in accordance with particular embodiments, the compound having structure (I) has the following structure (Id) (referred to herein as “Compound Id”):

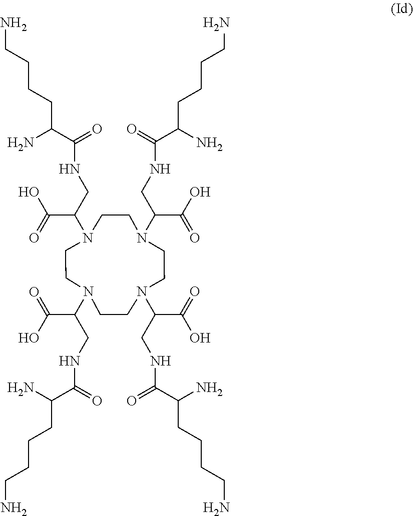 [SMILES: NCCCCC(N)C(=O)NCC(C(=O)O)N1CCN(C(CNC(=O)C(N)CCCCN)C(=O)O)CCN(C(CNC(=O)C(N)CCCCN)C(=O)O)CCN(C(CNC(=O)C(N)CCCCN)C(=O)O)CC1]

Embodiments of the invention also relate to a “complex” of a compound having structure (I) in accordance with any of the embodiments described above (e.g., Compound Ia, Ib, Ic or Id) with a metal. Such a complex is also referred to herein as a “contrast agent complex”. According to preferred embodiments, the complex is a “gadolinium complex,” i.e., the compound having structure (I) (e.g., Compound Ia, Ib, Ic or Id) is complexed with gadolinium (Gd). According to preferred embodiments, the complex has an overall net positive charge and is capable of interacting electrostatically with charged glycosaminoglycans in a subject.

According to one embodiment, the complex of the present invention has the following structure (A) (referred to herein as “Complex A”), which has a net positive charge of +2:

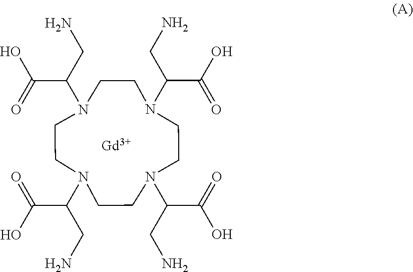

According to additional embodiments, the present invention relates to a contrast agent composition (also referred to as a contrast agent medium) comprising a contrast agent complex of the present invention in accordance with any of the embodiments described above, wherein the contrast agent complex is provided in a suitable carrier (preferably a liquid carrier) for administration to a subject. As used herein, the term “carrier” refers to a diluent, adjuvant, excipient, or vehicle with which a complex of the present invention is administered to a subject. Such carriers are preferably liquids; for example, saline, citrate buffer, phosphate buffered saline, HEPES ((4-(2-hydroxyethyl)-1-piperazineethanesulfonic acid) buffer or Tris buffer are preferred carrier(s). A contrast agent composition of the present invention may also include one or more additives, such as one or more of the following: wetting agents, excipients, emulsifying agents, pH buffering agents, antibacterial agents, antioxidants, chelating agents, etc. According to particular embodiments, a method for making a composition of the present invention comprises combining (e.g., mixing or suspending) a complex of the present invention with a carrier and one or more optional additives according to known methods.

The terms “subject” and “patient” are used interchangeably herein and refer to a mammalian individual, such as a mouse, rabbit, or human being. In pre-clinical settings, for example, a contrast agent composition of the present invention may be administered to a mouse or rabbit; in clinical settings, the contrast agent composition is preferably administered to a human being.

According to embodiments of the present invention, a method of using a contrast agent complex (or contrast agent composition) described herein comprises administering the contrast agent complex (or contrast agent composition) to a subject. For example, the contrast agent complex (or contrast agent composition) may be injected into a subject's joint (e.g., a knee joint, hip joint, ankle joint, wrist joint, elbow joint, shoulder joint, etc.). The amount of the contrast agent complex that is administered to the subject can be readily determined by one of ordinary skill in the art. For example, the contrast agent complex can be administered to a subject in an amount of between about 0.00001 mmole/kg and about 1 mmole/kg, or between about 0.0003 mmole/kg and about 0.3 mmole/kg, or between about 0.0001 mmole/kg and about 0.1 mmole/kg.

According to particular embodiments, the method further comprises imaging the subject; for example, imaging the subject's joint by using computed tomography (CT) and/or magnetic resonance imaging (MRI) according to known methods. According to an exemplary embodiment, the contrast agent complex of the present invention is used to perform magnetic resonance arthrography (MRA) to image a subject's joint. In accordance with this embodiment, a suitable amount of the contrast agent complex (or contrast agent composition) is injected into a subject's joint, and then imaged by magnetic resonance imaging (MRI).

According to particular embodiments, contrast agent complexes of the present invention improve the ability to detect soft tissue defects (e.g., tears in cartilage or other soft tissue) by providing increased signal intensity along the length of a tear, particularly in comparison to a clinically employed contrast agent (Magnevist®, also known as Gd-DTPA).

The embodiments of the invention are described above using the term “comprising” and variations thereof. However, it is the intent of the inventors that the term “comprising” may be substituted in any of the embodiments described herein with “consisting of” and “consisting essentially of” without departing from the scope of the invention. Unless specified otherwise, all values provided herein include up to and including the starting points and end points given.

The following examples further illustrate embodiments of the invention and are to be construed as illustrative and not in limitation thereof.

### EXAMPLES

### Example 1

As described below, the efficacy of Complex A in highlighting soft tissue tears was evaluated in comparison to a clinically employed contrast agent (Magnevist®) using explants obtained from adult bovine menisci, a fibrocartilaginous tissue in the knee joint. Complex A appeared to improve the ability to detect soft tissue defects by providing increased signal intensity along the length of the tear. Magnevist® showed a strong signal near the liquid-meniscus interface, but less contrast was observed within the defect at greater depths compared to what was observed with Complex A.

Compound Ia was synthesized as shown below:

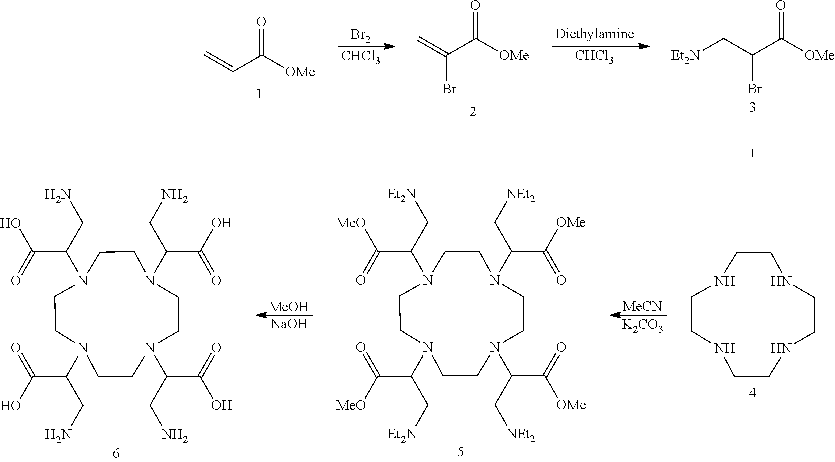

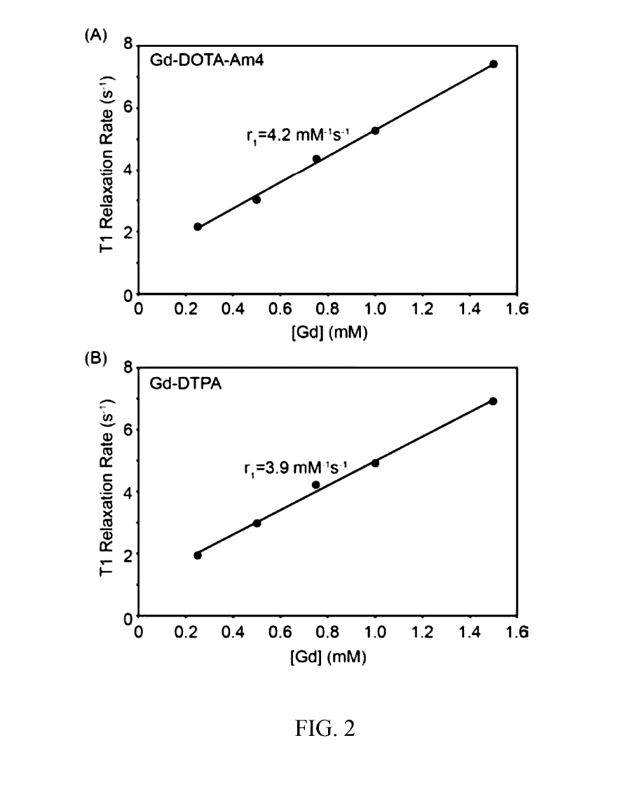

Compound2 was prepared by bromination of methyl acrylate using bromine in chloroform at room temperature, while compound 3 was prepared by slow addition of diethylamine into compound 2 in ether at 0° C. The methyl and ethyl protecting groups were efficiently removed with a methanol:NaOH (0.5 M) solvent cocktail (40:60) for 48 h. The solution was neutralized with a 2 M HCl solution and dried. The residue was redissolved in pure methanol, and the precipitate that formed was filtered and the solvent dried in vacuuo to yield compound 6. Compound 6 (referred to as Compound Ia) was then complexed with gadolinium (Gd) to form Complex A. Following complexation with Gd, it was determined that Complex A possessed a longitudinal relaxivity of 4.2 mM⁻¹s⁻¹ (FIG. 2A), using a Bruker mq60 MR relaxometer operating at 1.41 T (60 MHz). In comparison, the longitudinal relaxivity of Magnevist® was measured to be 3.9 mM⁻¹s⁻¹ (FIG. 2B).

To directly compare the stability of Complex A to Gd-DTPA (Magnevist®) and Gd-DOTA (also known as gadoteric acid), a transmetallation assay using equimolar concentrations of ZnCl₂ was performed in fetal bovine serum. Transmetallation was monitored as a function of time by calculating the relaxation ratio, R1(t)/R1(0), where R1(t) is the longitudinal Relaxivity at time t and R1(0) is the longitudinal Relaxivity in the absence of ZnCl₂ (i.e. time 0).

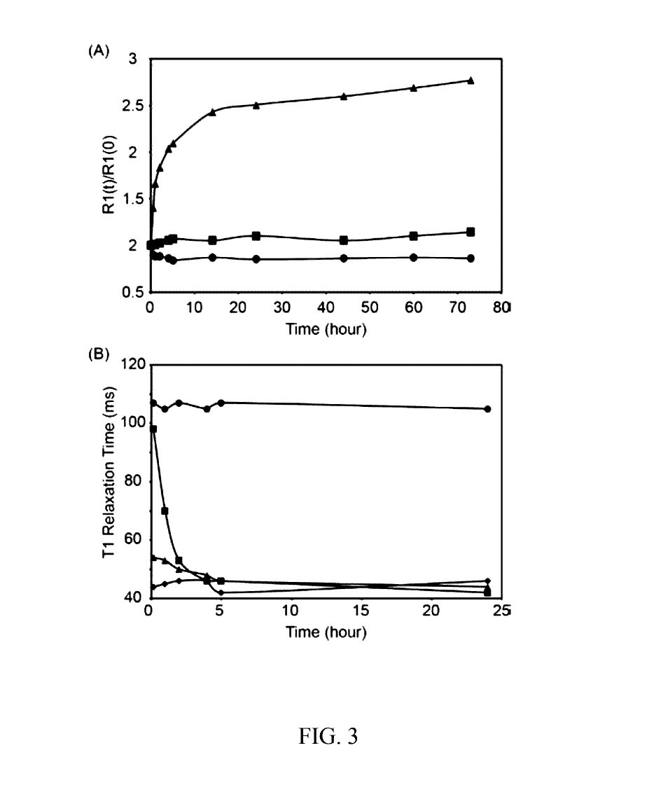

Consistent with previous reports, the relaxation ratio R1(t)/R1(0) rapidly increased for samples containing Gd-DTPA, indicative of transmetallation (FIG. 3A). No transmetallation was detected in samples containing Gd-DOTA or Complex A over the course of 72 hours.

Since transmetallation was not observed in serum supplemented with ZnCl₂, the stability of the various Gd complexes were further evaluated by monitoring demetallation in a highly acidic, non-physiologic solution (1M HCl). Even under these harsh conditions, Gd remained complexed with Compound 6 for at least 24 hours (FIG. 3B). In contrast, both Gd-DTPA and Gd-DOTA exhibited rapid demetallation. Since a measurement of the T1 relaxation time could not be acquired at t=0 for this assay, the relaxation ratio could not be calculated. Instead, demetallation was monitored strictly by changes in the T1 relaxation time. In instances of demetallation, the T1 relaxation time moved towards that of free Gd.

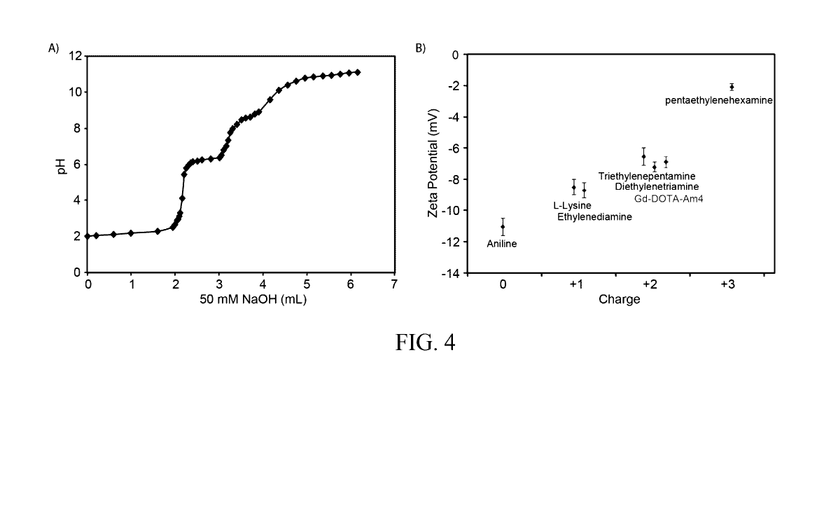

A pH-titration was carried out for Gd-DOTA-Am4 (FIG. 4A). The pKa values of Gd-DOTA-Am4 were extracted from the data and determined to be 2.1, 6.2, and 8.4. Zeta potential measurements revealed that Gd-DOTA-Am4 possessed a 2+charge at physiological pH (FIG. 4B). The zeta potential of Gd-DOTA-Am4 was compared with the zeta potential of various compounds with known charges at pH 7.4, and Gd-DOTA-Am4 had a zeta potential similar to that of other compounds with a +2 charge.

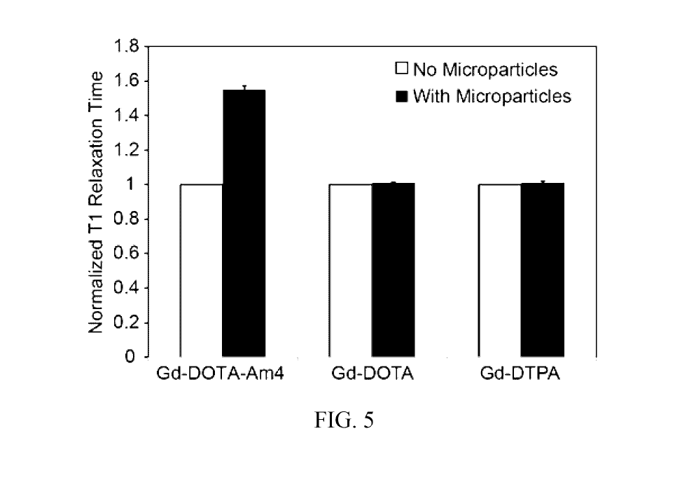

To assess whether Gd-DOTA-Am4 interacts electrostatically with GAG-rich cartilage, articular cartilage from juvenile bovine femurs was ground into microparticles. These microparticles were then mixed and incubated with Gd-DOTA, Gd-DTPA, or Gd-DOTA-Am4. The microparticles were then pelleted and the T1 relaxation time of the supernatant was measured. Measurements were compared to analogous control Gd complexes that were not incubated with cartilage microparticles (FIG. 5). All samples were normalized to the average T1 relaxation time of their respective control. Only the supernatant of the cationic Gd-DOTA-AM4 sample that was mixed with microparticles had a longer T1 relaxation time than the analogous sample without microparticles. This finding is consistent with our hypothesis that Gd-DOTA-Am4 interacts electrostatically with the negatively charged cartilage and as a result was partially depleted from the supernatant, resulting in a longer T1 relaxation time. The other Gd complexes, which are both anionic, did not interact with the cartilage microparticles and thus remained entirely in the supernatant, leading to no change in the relaxation time.

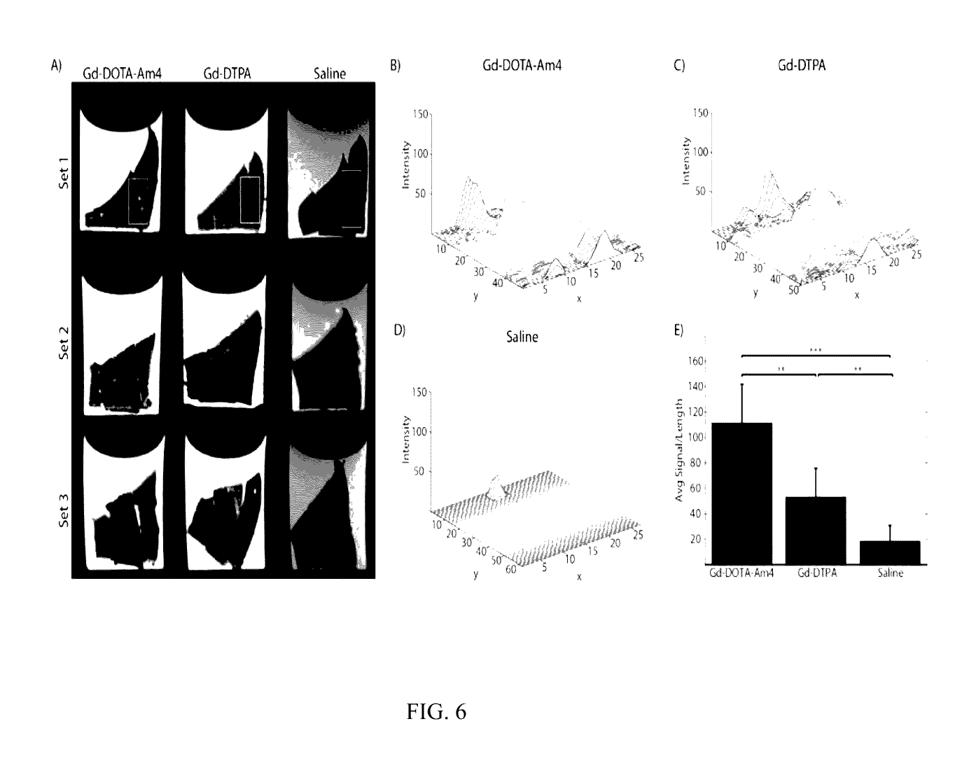

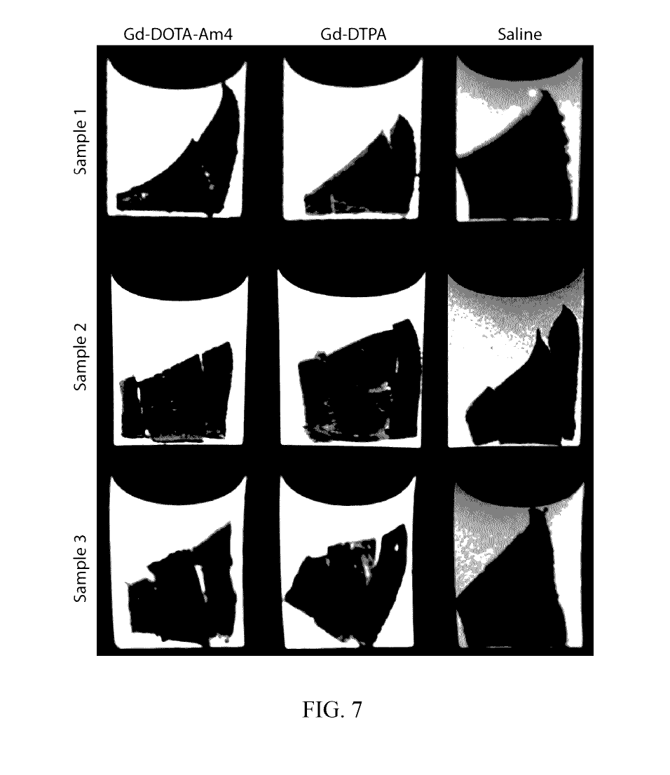

To evaluate the efficacy of Complex A in highlighting soft tissue tears in comparison to Gd-DTPA, explants (8 mm in diameter) were obtained from adult bovine menisci. Well-defined defects were then introduced by using a punch to create a 4 mm internal core, which was left in place. Tissue explant blocks (n=9; 3 per group) were placed in a 48-well microplate and bathed in 2 mM Gd-DTPA, Complex A, or Saline (pH 7.4) for ˜30 min. The pH and concentration of Gd⁺³ was kept constant throughout the experiment. T1-weighted images were then acquired in the axial plane (FIGS. 6A and 7). Qualitatively, Complex A appeared to improve the ability to detect the soft tissue defect by providing increased signal intensity along the length of the tear. Gd-DTPA led to a strong signal near the liquid-meniscus interface, but much less contrast was observed within the defect at greater depths. In the saline samples, the defects were extremely difficult to identify. To acquire a more quantitative comparison between the three groups, regions of interest (ROI) were drawn around the lateral defects and, following background subtraction (FIGS. 6B-D), the total signal intensity (area under curve) within the ROI was quantified and normalized to the length of the tear (FIG. 6E). The Wilcoxon signed-rank test was then used to compare contrast enhancement for the various contrast agents. It was determined that Complex A led to a statistically significant improvement in contrast along the defects compared with Gd-DTPA and saline. Gd-DTPA led to a statistically significant improvement in contrast compared to saline.

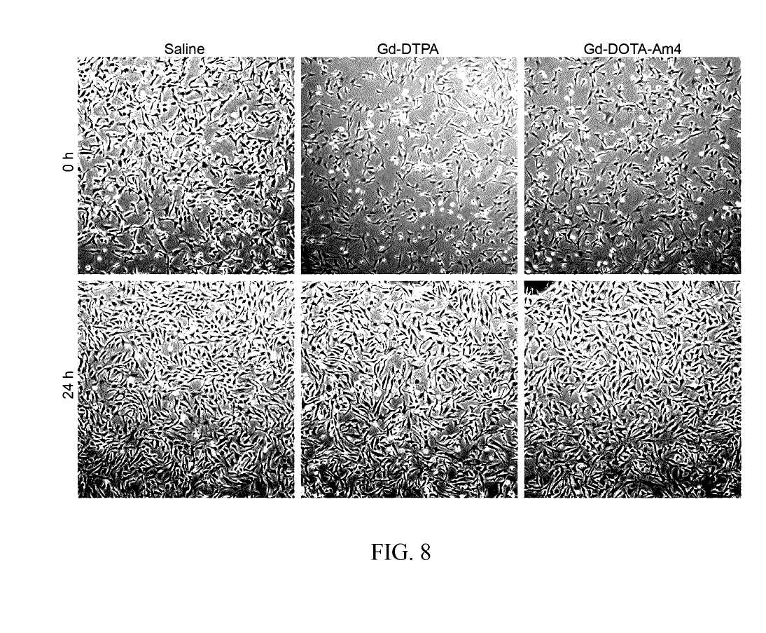

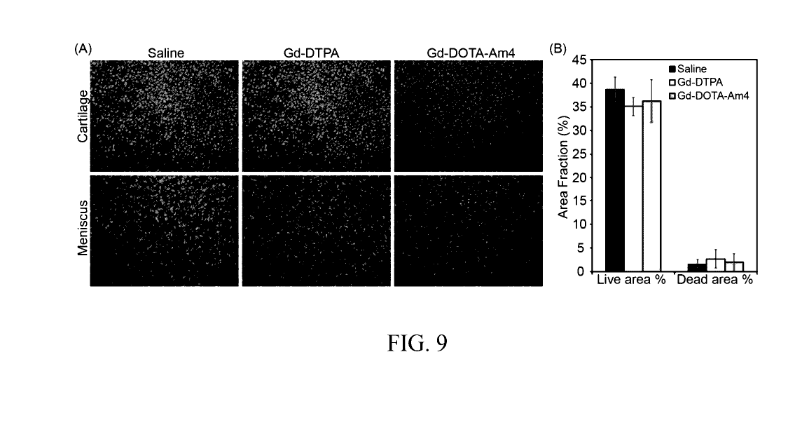

The safety profile of Gd-DOTA-Am4 was assessed via both cell- and tissue-based assays. When monolayers of isolated bovine meniscal fibrochondrocytes (MFCs) were exposed to basal media containing Saline, Gd-DTPA, or Gd-DOTA-Am4, cell morphology, viability, and proliferation over 24 hours were qualitatively similar between groups (FIG. 8). Similarly, after intra-articular injection of Saline, Gd-DTPA, or Gd-DOTA-Am4 into the joint space of an intact bovine femorotibial explant, the area fractions of live and dead cells in the cartilage and meniscus were equivalent after 3 hours (p>0.05) (FIG. 9).

The data presented here indicate that positively charged contrast agents can improve the identification of tears in the knee meniscus, and likely other GAG-rich cartilaginous tissues of the major joints. Complex A is also expected to allow for the improved assessment of cartilage degeneration, cartilage thickness, morphology, or GAG content. Notably, no significant side effects are expected owing to the low Gd dose needed to fill the joint space, the high stability of the cationic agent, and the strong safety profile of other Gd-DOTA-based agents following intra-articular administration.

### Materials and Methods

The following materials and methods were used to carry out Example 1 above.

Materials

Compound 1 and bromine were purchased from Fisher Scientific (Philadelphia, Pa.) and 1,4,7,10-tetraazacyclododecane (4) was purchased from Strem Chemical (Newburyport, Md.). Diethylenetriaminepentaacetic acid (DTPA) gadolinium complex was purchased from Aldrich (St. Louis, Mo.) while 1,4,7,10-tetraazacyclododecane-1,4,7,10-tetraacetic acid (DOTA) gadolinium complex was purchased from Macrocyclics (Dallas, Tex.). HyClone® Fetal Bovine Serum was purchased from GE Healthcare Life Sciences (Logan, Utah).

Instrumentation and Services

Elemental analysis was performed on C, H, and N by Intertek (Philadelphia, Pa.). 1H NMR spectra were acquired with a Bruker Avance-360 spectrometer. The relaxometric studies were performed using a Bruker mq60 tabletop MR relaxometer operating at 1.41 T.

Syntheses of Ligand and Contrast Agents.

Methyl α-bromoacrylate (Compound 2): Compound 2 was prepared as reported in Rachon, J.; Goedken, V.; Walborsky, H. M., Rearrangement of a bicyclic [2.2.2] system to a bicyclic [3.2.1] system. Nonclassical ions. J. Org. Chem. 1989, 54, 1006-1012.

Methyl α-bromo-β-diethylaminopropionate (Compound 3): To a stirred solution of compound 2 (19.1g; 0.11 mol) in 200 mL of diethylether at 0° C. was slowly added diethylamine (7.81 g; 11.0 mL; 0.011 mol). The solution was stirred for 3 h at room temperature and dried under vacuum. The resulting brown liquid was used immediately for the next step.

1,4,7,10-tetraazacyclododecane-N,N′,N″,N′″-(α-(diethylaminomethyl))-tetraacetic acid, methyl ester (Compound 5): 1,4,7,10-tetraazacyclododecane (Compound 4; 1.0 g; 5.81 mmol), anhydrous potassium phosphate, tribasic, (5.0 g; 23.2 mmol) and compound 3 (5.85 g; 23.2 mmol) in 150 mL of dry acetonitrile were stirred at 60° C. for 48 h. The solid base was filtered and the solvent was dried under vacuum. The title compound was purified by chromatography on silica column (chloroform:methanol 7:1) to give final product (45%). m/z (ESI); 801 (M+H). Elemental analysis: (C₄₀H₈₀N₈O₈.HBr) calculated C 54.6, H 9.19, N 12.71; found C 54.81, H 9.07, N 12.93. ¹H NMR (360 MHz, CDCl₃) δ 0.8-1.5 (12H, —CH₃, m), 2.4-2.8 (8H, —CH₂—, m, br), 2.9-3.0 (8H, —CH₂N—, m, br), 3.1-3.3 (8H, —NCH₂CH₂N—, m), 3.3-3.5 (4H, —CH—, m), 3.6-3.8 (12H, —OCH₃, br).

1,4,7,10-tetraazacyclododecane-N,N′,N″,N′″-(α-(aminomethyl))-tetraacetic acid (Compound 6 (also referred to as Compound Ia)): Compound 5 (1.0 g; 1.3 mmol) was stirred in 100 mL of methanol:1:0.5M NaOH (40:60) at room temperature for 48 h. The reaction mixture was neutralized with 2M HCl and dried under vacuum. The solid residue was re-dissolved in methanol, filtered and the solvent was removed under vacuum to yield the title compound (60%). m/z (ESI); 261 (M+2H). Elemental analysis: (C₂₀H₄₀N₈O₈. NaBr) calculated C 54.6, H 9.19, N 12.71; found C 54.81, H 9.07, N 12.93. ¹H NMR (360 MHz, (CD₃)₂SO) δ 1.0-1.4 (8H, —CH₂—, t), 2.7-2.9 (4H, —CH—, q), 3.0-3.8 (8H, —NCH₂CH₂N—, m, br), 8.8-9.1 (8H, —NH₂, s, br).

Complex A (also referred to herein as “Gd- DOTA-Am4”) and Gd-DTPA. The complexes were synthesized as previously described in Nwe, K.; Bryant, L. H.; Brechbiel, M. W. Poly(amidoamine) Dendrimer Based MRI Contrast Agents Exhibiting Enhanced Relaxivities Derived via Metal Preligation Techniques. Bioconjugate Chem. 21, 1014-1017. Gadolinium concentration was determined by ICP-OES analysis using an Elan 6100 ICP-MS (Perkin-Elmer, Shelton, Conn.) at the New Bolton Center Toxicology Laboratory, University of Pennsylvania, School of Veterinary Medicine, Kennett Square, Pa., USA.

Relaxivity Measurements

Longitudinal relaxation times (T1) were measured in saline at pH 7.4 using a Bruker mq60 tabletop MR relaxometer operating at 1.41 T. The longitudinal relaxivity (r1) of the complex was calculated by plotting the reciprocal of the T1 relaxation time versus the gadolinium concentration.

Transmetallation Assay

Transmetallation of Gd³⁺ ion by Zinc²⁺ ion was performed in fetal bovine serum. The solution contained 4 mL of 2.5 mM Gd-complex and 2.5 mM ZnCl₂ at pH 7.4. The mixture was stirred at 37° C. throughout the experiment while 300 μL aliquot was taken up at the appropriate time for measurement of the longitudinal relaxation time. The water proton relaxation times were measured using a Bruker mq60 tabletop MR relaxometer operating at 1.41T. Ratio of reverse relaxation time at each time point (R1(t)) and relaxation time at time zero (R1(0)) was plotted against time.

Demetallation Assay A 300 μL solution of 2.5 mM Gd-compound was incubated at 37° C. in 1M HCl and the longitudinal relaxation times were acquired at appropriate time points. The relaxation time was then plotted against time.

Preparation of Menisci Samples for Imaging

Adult bovine stifle joints were purchased from a commercial vendor (Animal Technologies, Tyler, Tex.), dissected in a sterile field, and the menisci removed. Biopsy punches (Miltex, York, Pa.) were used to produce 8 mm diameter full thickness samples in the axial plane. Well-defined defects were then introduced by using a 4 mm punch to create an internal core, which was left in place. The meniscus samples were placed in a 48-well plate, bathed in 2 mM [Complex A]⁺³, [Gd-DTPA]⁻², or saline (pH 7.4) for 30 minutes and imaged via MR using a T1-weighted sequence.

Contrast-Enhanced MR Imaging

Magnetic Resonance images were acquired using a 4.7 T small animal horizontal bore Varian INOVA system. T1-weighted images were acquired in the axial plane using parameters as follows: repetition time (TR)=2000 ms, echo time (TE)=20 ms, FOV=40×40 mm, flip angle=90°, slice thickness=1.0 mm, number of requisition=2, matrix=256×256 pixels.

Image Analysis

A region of interest (ROI) was determined for each MR image slice (n=6 per contrast agent, i.e. 2 slices per sample). All ROIs had a width of 27 pixels, as it could include the entire tear for all slices. The ROI height was determined for each slice individually, such that it could span the sample from top to bottom and exclude media and unrelated bright areas within the cartilage (e.g., cavities near the liquid-cartilage interface). In samples where there was clearly a pool of solvent within the tear, the ROI spanned the longer of the two segments between the pool and the top or bottom of the cartilage.

ROIs were partitioned into one-pixel tall lines spanning the entire width. Line signal intensities were plotted against their position and the bottom 80% of values were used to determine a zero-intensity baseline for that line. The 80th percentile was used because it minimized the variance of baseline y-intercepts for the lines within each individual ROI. For each line, the trapezoid method was used to integrate the area under the curve. All of the integrations in an ROI were then averaged to quantify that ROI's average signal intensity. The t-test was used to determine significance (p<0.05) between the average signal intensity of the contrast agents.

### Example 2

Compound Ib (“DOTA-AmS4”) was synthesized as shown below:

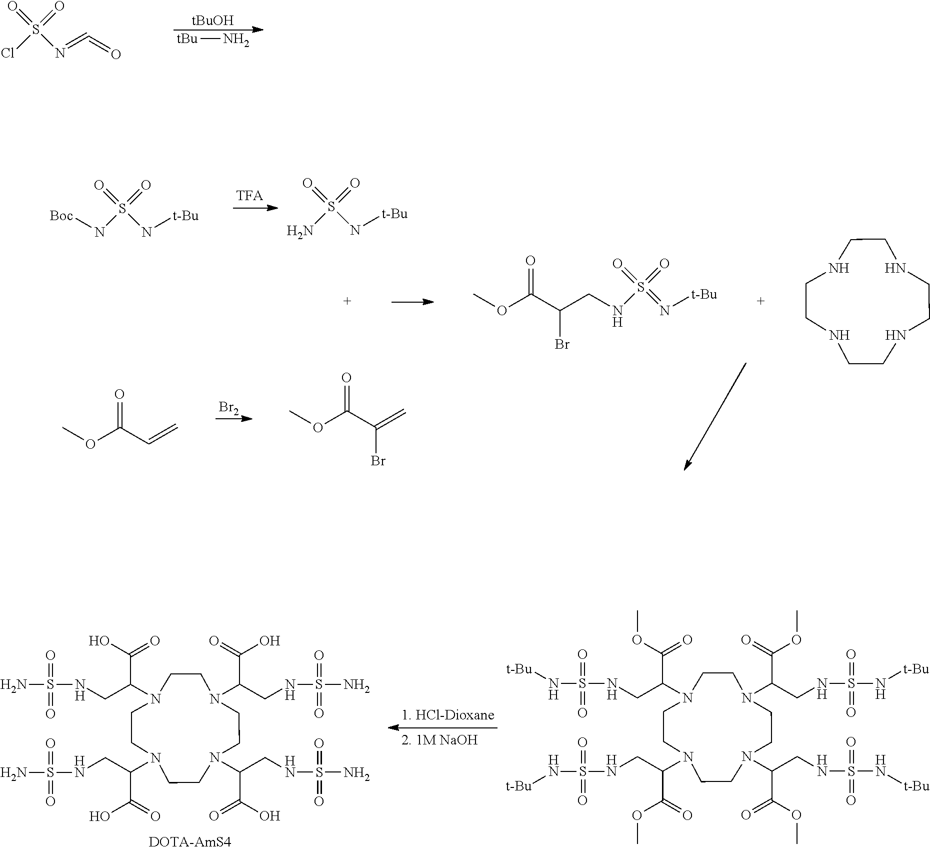

Compound Ib was prepared as follows: Chlorosulfonyl isocyanate was reacted with tert-butanol and tert-butylamine in toluene to yield tert-butyl N-tert-butylsulfamoylcarbamate followed by removal of Cert-butylcarbamate using trifluoroacetic acid. The resulting N-tert-bytulsulfamide was reacted with brominated methylacrylate followed by a subsequent reaction with 1,4,7,10-tetraazacyclododecane (cyclen). The tert-butyl groups were removed via treatment with 2M hydrochloric acid in dioxane (2M-HCl-Dioxane) while methyl groups were removed by treatment with 1M NaOH to yield compound Ib.

Compound Ic (“DOTA-Ac4”) was synthesized as shown below:

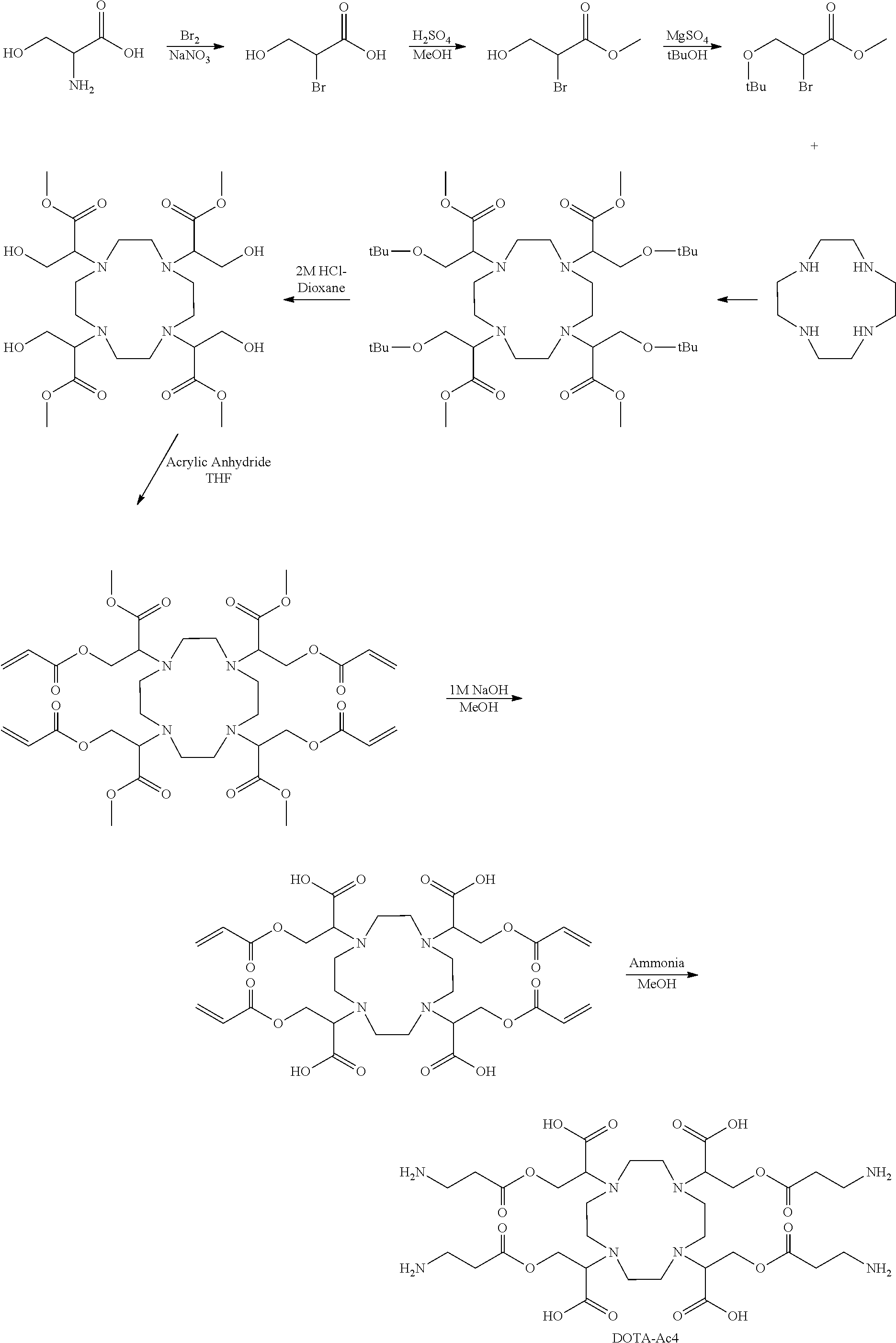

Compound Ic was prepared as follows: L-serine was brominated using bromine and sodium nitrite, followed by protection of carboxylates using methanol with a catalytic amount of sulfuric acid. The alcohol was protected with tert-butyl group using magnesium sulfate and tert-butanol. The resulting compound was allowed to react with cyclen followed by deprotection of tert-butyl groups using 2M HCl-Dioxane. The resulting compound with free alcohol was then reacted with acrylic anhydride followed by deprotection of methyl groups using 1M NaOH. Amination reaction was done by using 1M ammonia in methanol solution to yield compound Ic.

Compound Id (“DOTA-Lys4”) was synthesized as shown below:

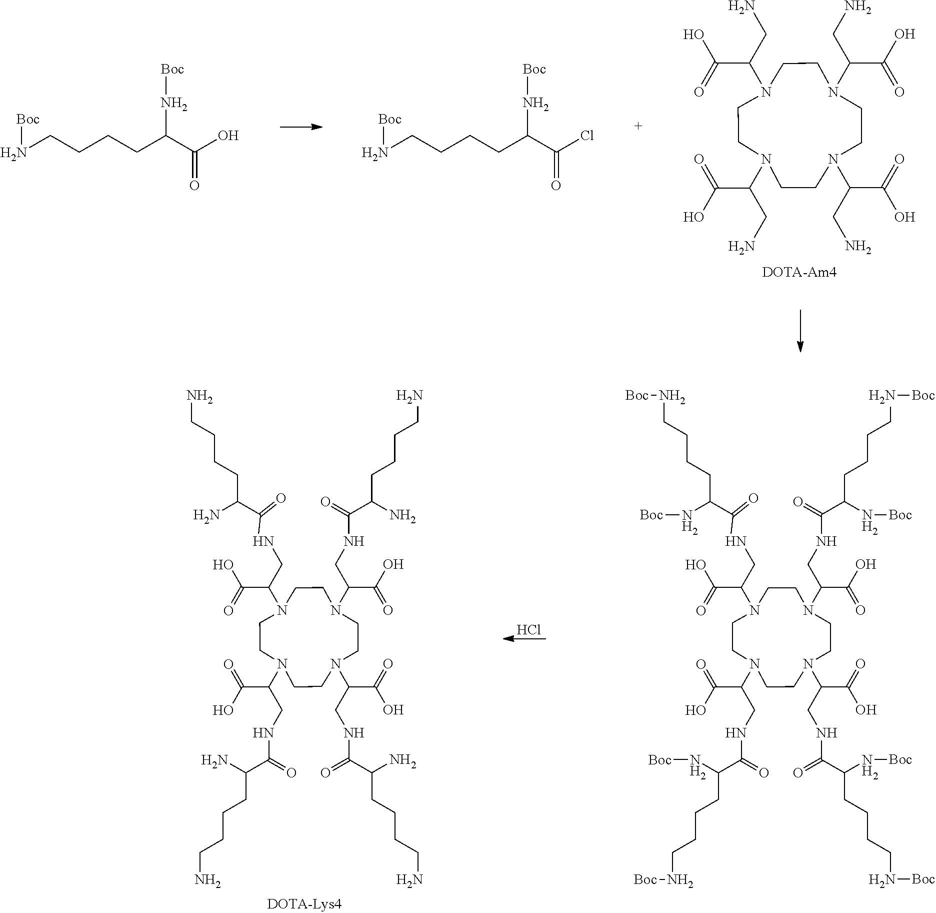

Compound Id was prepared as follow: Tert-butyloxycarbonyl (Boc)-protected lysine (Chem Impex Int'l Inc., Wood Dale, Ill.) was chlorinated using thionyl chloride in benzene. The resulting compound was reacted with DOTA-Am4 followed by deprotection of Boc-protecting groups using HCl to yield compound Id.

The embodiments described herein are intended to be exemplary of the invention and not limitations thereof. One skilled in the art will appreciate that modifications to the embodiments and examples of the present disclosure may be made without departing from the scope of the present disclosure.

What is claimed is:

1. A polyazamacrocyclic compound having structure (I): 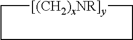
where each x is 2 or 3, y is 3 or 4, R is —CH(CO₂X)R′, X is H or an alkali metal, and R′ is a primary amine-functionalized substituent.

2. The compound of claim 1, wherein the primary amine-functionalized substituent contains two or more primary amine groups.

3. The compound of claim 1, wherein the primary amine-functionalized substituent contains a primary amine group at the terminus of an organic moiety.

4. The compound of claim 3, wherein the organic moiety contains a backbone chain of from 1 to 10 atoms.

5. The compound of claim 4, wherein the backbone chain contains at least one carbon atom and, optionally, at least one heteroatom selected from S, O or N.

6. The compound of claim 1, wherein the primary amine-functionalized substituent corresponds to structure (II):
—CH₂(R″)NH₂ (II)
wherein R″ is a covalent bond or a divalent organic moiety.

7. The compound of claim 6, wherein the divalent organic moiety contains a backbone chain of from 1 to 9 atoms.

8. The compound of claim 7, wherein the backbone chain consists of atoms selected from the group consisting of C, S, O and N.

9. The compound of claim 6, wherein R″ is a covalent bond.

10. The compound of claim 6, wherein R″ is —NHSO₂—, —OC(═O)CH₂CH₂—, or —NHC(═O)CH(NH₂)—(CH₂)₄—.

11. The compound of claim 1, wherein x is 2 and y is 4.

12. A contrast agent complex of a compound having structure (I), in accordance with claim 1, with a metal.

13. The contrast agent complex of claim 12, wherein the metal is gadolinium.

14. The contrast agent complex of claim 12, wherein the complex has an overall net positive charge.

15. The contrast agent complex of claim 12, wherein the complex is selected from the group consisting of Compound Ia complexed with gadolinium, Compound Ib complexed with gadolinium, Compound Ic complexed with gadolinium and Compound Id complexed with gadolinium.

16. A contrast agent composition comprising the contrast agent complex of claim 12, a carrier, and one or more optional additives.

17. A method of using the contrast agent complex of claim 12 comprising administering the contrast agent complex to a subject.

18. A method of using the contrast agent complex of claim 12 comprising injecting the contrast agent complex into a subject's joint.

19. The method of claim 17 further comprising imaging the subject's joint by computed tomography or magnetic resonance imaging.

20. The method of claim 17 comprising performing magnetic resonance arthrography to image the subject's joint.
